(19) United States
(12) Patent Application Publication
(10) Pub. No.: US 2026/0325124 A1
(43) Pub. Date: Oct. 22, 2026

(54) IMPLANTATION SET

(71) Applicant: KAIROS MEDICAL AG, Bettlach (CH)

(72) Inventors: Leopold BERGER, Zürich (CH); Sandro MATTER, Gipf-Oberfrick (CH)

(21) Appl. No.: 19/182,071

(22) Filed: Apr. 17, 2025

(51) Int. Cl. A61B 17/04; A61B 17/00; A61B 17/68; A61B 17/86; A61F 2/08; A61L 33/02; A61L 33/06

(52) U.S. Cl. CPC A61B 17/0401; A61B 17/8605; A61B 17/866; A61F 2/0811; A61L 33/022; A61L 33/025; A61L 33/064; A61B 2017/00836; A61B 2017/00929; A61B 2017/0409; A61B 2017/0414; A61B 2017/044; A61B 2017/0445; A61B 2017/0464; A61B 2017/681; A61F 2002/0852; A61F 2002/0858; A61F 2002/0888

(57) ABSTRACT

The present invention relates to an implantation set comprising an insertion device, an implant device, and one or more insulating member(s), the insertion device including an insertion bar extending along a longitudinal axis from a proximal end to a distal end and being configured for inserting the implant device into bone; the implant device including an implant head made from magnesium or a magnesium alloy and being releasably connectable to the distal end of the insertion bar, thereby bringing the insertion device and the implant device temporarily into a connected state, the insulating member including one or more head insulating member(s), thereby at least partially interrupting direct electric contact between the implant head and the insertion bar.

### FIELD OF THE INVENTION

The present invention relates to an implantation set comprising an insertion device, an implant device, and one or more insulating member(s) according to claim 1.

### BACKGROUND OF THE INVENTION

In contemporary minimally invasive orthopedic procedures, various kinds of implants are utilized, e.g. for reattaching soft tissues, such as tendons and ligaments, to their respective bony structures, or for stabilizing and compressing bone fragments to encourage proper healing. To facilitate the insertion of such implants, implantation sets have been developed that typically include an insertion bar featuring a shaft, designed to drive an implant head into the bone with precision and minimal exertion.

A specific class of implants that are inserted into a patient's body for reattaching soft tissues to bone are suture anchors. Suture anchors generally include an implant head that comprises one or more suture retention features such as eyelets, loops or slots, designed to hold and secure sutures. In case of so-called knotless suture anchor devices, an implant head with an eyelet arranged at a distal end of the shaft of an insertion bar may receive a loop of suture therethrough. An anchor sleeve with an outer thread can then be used to secure the implant head with the suture in the bone. The soft tissue is tensioned as the anchor sleeve and the implant head, respectively, are inserted into the bone to the appropriate depth.

In orthopedic procedures, the use of materials with sufficient stability and high biocompatibility is critical for successful patient outcomes and recovery. While the preferred material for the insertion bar is surgical steel, various materials that degrade over time have been evaluated for the implant parts remaining in the body after the surgical procedure. For instance, magnesium-based materials have been found to provide a significant potential in terms of mechanical stability, biodegradability, and osteogenic activities. However, corrosion processes taking place when in contact with bodily fluids and associated hydrogen gas release as a reaction product represent limitations which make the clinical application of magnesium-based implant parts challenging.

### SUMMARY OF THE INVENTION

It is therefore an object of the invention to provide an implantation set including an implant device made of magnesium or a magnesium alloy that avoids or at least reduces the occurrence of corrosion processes observed during implantation of magnesium-based implants described in the prior art.

According to the present invention, this object is achieved by the features of the independent claims. In addition, further advantageous embodiments are subject to the dependent claims.

According to one aspect, the invention provides an implantation set comprising an insertion device, an implant device, and one or more insulating member(s). The insertion device includes an insertion bar extending along a longitudinal axis from a proximal end to a distal end and is configured for inserting the implant device into bone. The implant device includes an implant head made from magnesium or a magnesium alloy and is releasably connectable to the distal end of the insertion bar, thereby bringing the insertion device and the implant device temporarily into a connected state. The insulating member includes one or more head insulating member(s) arranged between the insertion bar, specifically the distal end of the insertion bar, and the implant head in the connected state, thereby at least partially interrupting direct electric contact between the implant head and the insertion bar.

The inventive implantation set is particularly well suited for use in arthroscopic minimally invasive procedures, e.g. in the field of rotator cuff repair where torn tendons in the shoulder are repaired. In order to provide a clear view for a camera of an arthroscope, the surgical site may constantly be flushed with an irrigation liquid such as a NaCl solution. It was found that for an implantation set with an insertion bar being made from a material different than the implant head, such as surgical stainless steel, galvanic corrosion of the magnesium-based implant device may occur due to direct electric contact of the insertion bar with the implant head, effectively forming a galvanic cell in the environment of the irrigation liquid. Direct electric contact of the insertion bar with the implant head may, in particular, be enhanced by the high mechanical loads being transferred by the insertion bar and/or tight fits between the insertion bar and the implant head. Galvanic corrosion of the magnesium-based implant head may in turn cause undesired gas accumulation and bubble formation during arthroscopic insertion.

By providing an insulating member with at least one head insulating member arranged between the insertion bar, specifically the distal end of the insertion bar, and the implant head in the connected state direct electric conduction therebetween can be reduced or avoided and the effective formation of a galvanic cell be mitigated or prevented. The head insulating member is configured to at least partially interrupt direct electric contact between the implant head and the insertion bar. It may therefore be an electrically insulating part that galvanically isolates the implant head and the insertion bar such that galvanic corrosion of the magnesium-based implant head can significantly be reduced or avoided, allowing to mitigate undesired effects originating from galvanic corrosion of the implant head such as gas bubble formation.

The insertion bar may have a circular, partially circular or polygonal cross-section. In particular, the insertion bar may have axial portions with different cross-sections. For example, the insertion bar may have a first axial portion with a circular cross-section and a second axial portion with a polygonal, for example square or hexagonal, cross-section. The polygonal cross-section may be regular or irregular. In some variants, the cross-section of the insertion bar may partially be circular and partially polygonal.

In some embodiments, the head insulating member or at least one of the insulating members is made from one of: a polymer, polyether ether ketone, rubber, a ceramic material. The head insulating member may therefore be made from a non-conductive material allowing to at least partially interrupt electric contact between the implant head and the insertion bar.

In some embodiments, the head insulating member or at least one of the insulating members comprises an electrically insulating coating applied to a portion of the insertion bar interfacing the implant head and/or a portion of the implant head interfacing the insertion bar, in particular at least a portion of the distal end of the insertion bar.

In particular, the electrically insulating coating may be applied to the distal end of the insertion bar interfacing the implant head.

An electrically insulating coating may be used as an effective way to at least partially interrupt direct electric contact between the implant head and the insertion bar, as the relevant parts of the insertion bar and implant head that may come into direct contact with one another during arthroscopic insertion may selectively be covered by the coating. Furthermore, the electrically insulating coating provides enhanced flexibility as the coating may selectively be applied to a portion of the implant head or to a portion of the insertion bar or, if desired, to both a portion of the implant head and a portion of the insertion bar.

In a variant, the electrically insulating coating is applied to at least 80% of the surface of the insertion bar interfacing the implant head, or at least 80% of the surface of the implant head interfacing the insertion bar.

Preferably, the electrically insulating coating is applied to all the surface of the insertion bar interfacing the implant head, or all the surface of the implant head interfacing the insertion bar.

Covering all the interfacial surface with an insulating coating ensures an optimal insulation effect.

In some embodiments, the electrically insulating coating is a biocompatible polymer or lacquer. Preferably, the electrically insulating coating is selected from the group consisting of aluminum oxide, zirconia, yttria-stabilized zirconia, chromium oxide, titanium dioxide, magnesium aluminate spinel, hafnium oxide, lanthanum-based oxides, calcium zirconate, silicon oxide, hydroxyl apatite, a lubricant varnish, parylene, diamond-like carbon, an electrolytic oxidized plasma coating, and combinations thereof. These coatings prevent galvanic corrosion, thereby reducing the risk of bubble formation during insertion. Furthermore, depending on the material chosen, they can serve a dual purpose, for example, by additionally promoting a biological response or providing a low-friction surface.

It has been demonstrated that the insertion bar made of surgical steel or titanium or a titanium alloy and the implant head made of magnesium or magnesium alloys or parts of the insertion bar or implant head thereof can be effectively coated with the aforementioned coatings without negatively impacting the mechanical properties of the insertion bar or the implant head. On the contrary, the excellent properties can even be further enhanced. The electrically insulating coating can withstand the high mechanical load applied during the press-fit of the implant head and the insertion bar. Furthermore, these biocompatible coatings have an excellent oxidation resistance and chemical resistance to various media including blood.

Preferably, the electrically insulating coating comprises aluminum oxide, for which excellent results were achieved. Aluminum oxide forms a stable, protective oxide layer and can effectively prevent galvanic corrosion, thereby minimizing the risk of gas accumulation and bubble formation during arthroscopic insertion. Furthermore, it reduces the risk of adverse reactions and promotes a favorable biological response.

Alternatively, the electrically insulating coating comprises hydroxyl apatite, which not only effectively prevents galvanic corrosion but also promotes bone bonding and integration with biological tissues.

Alternatively, the electrically insulating coating comprises lubricant varnish that can be applied easily and provides effective electrical insulation combined with and a low-friction surface.

Alternatively, the electrically insulating coating comprises parylene that has a high dielectric strength providing good electrical insulation in thin layers. Since it can be applied in very thin layers, it can be particularly utilized in geometrically complex areas of the implant head.

Alternatively, the electrically insulating coating comprises diamond-like carbon, which creates a robust protective layer that effectively mitigates galvanic corrosion. Additionally, it can withstand the significant mechanical loads that may be experienced during the press-fit of the implant head and the insertion bar.

Alternatively, if the insertion bar is made of titanium or a titanium alloy, the electrically insulating coating may be formed by plasma electrolytic oxidation forming a ceramic-like coating that prevents galvanic corrosion.

In some embodiments, the electrically insulating coating has a thickness between 3 μm and 150 μm.

A thickness of the coating in this range ensures a sufficient electrical insulation even under the high mechanical load applied during the press-fit of the implant head and the insertion bar, while at the same time maintaining a smooth fit between the implant head and the insertion bar.

Preferably, the electrically insulating coating has a thickness between 20 μm and 100 μm, more preferably between 30 μm and 50 μm.

In some embodiments, the electrically insulating coating comprises a plurality of pores, and the head insulating member further comprises a polymeric sealing on top of the electrically insulating coating.

The electrically insulating coating may comprise pores. By closing a plurality of these pores of the electrically insulating coating with a polymeric sealing, the electrical insulation ability of the head insulating member is improved compared to a coating without additional polymeric sealing. Such a polymeric sealing can be applied easily to a variety of electrically insulating coatings.

The polymeric sealing may comprise a biocompatible polymer, such as polydimethylsiloxane, polyethylene glycol, poly(L-lactic acid), polyurethane, polyether ether ketone.

Preferably, the polymeric sealing comprises parylene, for which excellent results were achieved.

Parylene can be applied easily by chemical vapor deposition (CVD) or tip coating, which allows coating of complex geometries of the insertion rod or implant head. Due to its high dielectric strength, a parylene sealing ensures good electrical insulation in thin layers.

In a preferred embodiment, a parylene sealing is used in combination with an aluminum oxide electrically insulating coating. This combination yields excellent results for preventing galvanic corrosion.

In some embodiments, the electrically insulating coating is applied to a radial surface portion of the insertion bar and/or to a radial surface portion of the implant head.

The electrically insulating coating may therefore provide interruption of direct electric contact at a position where particularly high mechanical loads may occur. High mechanical loads may for example occur due to a tight fitting between the implant head and the insertion bar. Parts of the implant head and/or the insertion bar with an enhanced probability of establishing a direct electric contact may therefore be protected by the electrically insulating coating.

In some embodiments, the implant device is selected from the group consisting of a suture anchor, a fixation pin, a punching tip, and a screw.

In a preferred embodiment, the implant device is a suture anchor for attaching soft tissue to bone. The term suture anchor hereby relates to an implant used to attach soft tissue to bone. Suture anchors are characterized by their mechanical performance (such as pull-out strength and fixation stability), material composition and properties (metallic, polymeric, bioresorbable), and biocompatibility.

Suture anchors are often used in arthroscopic minimally invasive procedures, where irrigation liquids such as a NaCl solution are used to flush the surgical site. Therefore, preventing galvanic corrosion by providing a head insulating member is especially beneficial in implantation sets comprising suture anchors.

In some embodiments, the insertion bar comprises a bar axial stop surface and the implant head comprises a head axial stop surface, wherein the head insulating member or at least one of the head insulating members is arranged between the bar axial stop surface and the head axial stop surface.

By arranging the head insulating member between the bar axial stop surface and the head axial stop surface, direct electric contact between the implant head and the insertion bar which may occur due to axial load during arthroscopic insertion may be reduced or prevented.

In some embodiments, the head insulating member arranged between the bar axial stop surface and the head axial stop surface comprises an electrically insulating coating applied to at least one of: the bar axial stop surface, the head axial stop surface.

In particular, the electrically insulating coating is selected from the group consisting of aluminum oxide, hydroxyl apatite, a lubricant varnish, parylene, diamond-like carbon, and a plasma electrolytic oxidized coating of the insertion bar or portions thereof, preferably aluminum oxide.

Preferably, the electrically insulating coating has a thickness between 20 μm and 150 μm, preferably between 30 μm and 100 μm, more preferably between 30 μm and 50 μm.

The head insulating member arranged between the bar axial stop surface and the head axial stop surface may comprise a polymeric sealing closing a plurality of pores of the electrically insulating coating. Preferably, the polymeric sealing comprises parylene.

In some embodiments, the head insulating member arranged between the bar axial stop surface and the head axial stop surface comprises a disc, preferably a disc with a central aperture. By providing a disc, the bar axial stop surface and the head axial stop surface can axially be spaced from each other. The central aperture may serve to receive a coupling structure therethrough such as a pin of the insertion bar or the implant head.

In some embodiments, the insertion bar comprises a bar pin at its distal end, preferably protruding from the bar axial stop surface, wherein the implant head comprises a head bore dimensioned to receive the bar pin, wherein, in the connected state, at least one head insulating member is arranged between the bar pin and a side wall of the head bore.

The implant head and the insertion bar can therefore be connected by the bar pin and the head bore. The head bore may have a circular or polygonal cross-section. The polygonal cross-section may be regular or irregular. In some variants, the head bore may have a partially circular and a partially polygonal cross-section. By providing a bar pin and a head bore, the region of possible direct contact between the insertion bar and the implant head can be confined, thereby facilitating interruption of direct electric contact of the insertion bar and the implant head. In some embodiments, the implant head comprises a head pin protruding from the head axial stop surface, wherein the insertion bar comprises a bar bore dimensioned to receive the head pin, wherein at least one head insulating member is arranged between the head pin and a side wall of the bar bore.

In some embodiments, the at least one head insulating member arranged between the bar pin and the side wall of the head bore comprises an O-ring, wherein the bar pin comprises at least one circumferential recess dimensioned to partially receive the O-ring.

The bar pin and the side wall of the head bore can therefore radially be spaced from each other by the O-ring positioned in the circumferential recess of the bar pin. Preferably, the bar pin comprises at least two circumferential recess such that at least two O-rings can be placed between the bar pin and the side wall of the head bore. Thus, an inexpensive and flexible interruption of direct electric contact between the insertion bar and the implant head can be achieved by using O-rings as a head insulating member.

In some embodiments with a head pin and a bar bore, the at least one head insulating member arranged between the head pin and the side wall of the bar bore comprises an O-ring, wherein the head pin comprises at least one circumferential recess dimensioned to partially receive the O-ring.

In some embodiments, the bar pin is dimensioned to engage with the head bore by a press fit. This means that the bar pin is inserted into the head bore with at least some pressure, creating a tight fit due to the precise matching of their dimensions. This usually involves slight interference where the bar pin is slightly larger than the bore, creating a secure, friction-based fit without the need for additional fastening components.

In some embodiments, the implant head is a screw head including a head bore having one or more planar radial stop surfaces, and the insertion bar or the bar pin comprises a distal end portion including engagement elements dimensioned to engage, i.e. to fit and interact, with the radial stop surfaces for transferring a torque from the insertion bar to the screw head.

In some embodiments, the insertion bar comprises an internal channel dimensioned to allow a suture (a thread used in medical procedures) to pass through it.

In some embodiments, the insertion bar comprises a distal end portion with a circular cross-section and the implant head comprises a bore having one or more planar radial stop surfaces dimensioned to engage with the distal end portion of the insertion bar by a press fit.

By providing one or more planar radial stop surfaces that engage with the circular distal end portion, detrimental peak loads at the occurrence of skewness or misalignment between the insertion bar and the implant head can be reduced or avoided, while providing a sufficient pull-off force (force required to separate the bar and head) between the insertion bar and the implant head. Detrimental peak loads may, in particular, be reduced compared to a press fit between a circular cross-section of the insertion bar end portion and a circular cross-section of the bore of the implant head.

In some embodiments, the bore of the implant head comprises four planar radial stop surfaces, wherein neighboring planar radial stop surfaces are perpendicularly oriented to each other. This rectangular configuration provides an optimal balance between skewness stability as described above and sufficient pull-off force.

In some embodiments, the implant head comprises a distal end portion with a circular cross-section and the insertion bar comprises a bore having one or more planar radial stop surfaces dimensioned to engage with the distal end portion of the implant head by a press fit.

In some embodiments, the bore of the insertion bar comprises four planar radial stop surfaces, wherein neighboring planar radial stop surfaces are perpendicularly oriented to each other. This configuration improves the interaction with corresponding parts, ensuring better alignment and stability.

In some embodiments, a head insulating member comprises a stop disc with a proximal circumferential surface (i.e. the surface of the stop disc closer to the insertion bar) forming a first axial stop surface for the insertion bar and a distal circumferential surface forming a second axial stop surface for the implant head. The axial stop surface thus prevents further movement in the axial (lengthwise) direction.

The insertion bar and the implant head may therefore axially be spaced from one another by the stop disc such that direct electric contact between the implant head and the insertion bar which may occur due to axial load during arthroscopic insertion may be reduced or prevented.

Thus, the stop disc, is used to create axial separation between the insertion bar and the implant head. This separation helps in maintaining the electrical isolation of the two components and it ensures that the components maintain their respective positions and integrity under axial loads.

In some embodiments, the head insulating member having the stop disc comprises a proximal pin proximally protruding from the stop disc, wherein the insertion bar comprises a bore dimensioned to receive the proximal pin protruding from the stop disc.

The head insulating member having the stop disc may therefore be secured at the insertion bar by the proximal pin.

In some embodiments, the proximal pin is engageable with the bore of the insertion bar by a press fit.

The tight fit created by a press-fit, also known as an interference fit, ensures that the components are securely joined together without the need for additional fasteners like screws, bolts, or adhesives. This results in a strong, reliable joint.

Also, because a press-fit creates a large contact area between the two components, it can effectively transfer loads (axial, radial, or torsional) between the components.

In some embodiments, the proximal pin comprises an outer thread and the bore of the insertion bar comprises an inner thread which is engageable with the outer thread of the proximal pin.

Thus, in such embodiments, the proximal pin and the insertion bar are connected using a threaded engagement. Specifically, the proximal pin has external threads, and the insertion bar has corresponding internal threads in its bore. When these threads are screwed together, they create a secure and stable connection. The threading helps in aligning the components precisely during assembly and the threads distribute the load over a larger surface area, reducing the stress on any one point and enhancing the durability of the joint.

In some embodiments, the head insulating member having the stop disc comprises a distal pin distally protruding from the stop disc, wherein the implant head comprises a head bore dimensioned to receive the distal pin protruding from the stop disc. The head insulating member having the stop disc may therefore be secured at the implant head by the distal pin.

In some embodiments, the distal pin is engageable with the head bore of the implant head by a press fit.

The implant head may therefore be secured to the insertion bar by a press fit with a portion of the insertion bar or by a press fit with a portion, such as a distal pin, of a head insulating member being secured at the insertion bar.

In some embodiments, the implant head may be secured to the insertion bar by a suture guided through an eyelet of the implant head.

In some embodiments, the press fit between the proximal pin of the head insulating member and the bore of the insertion bar has a larger tightness than the press fit between the distal pin of the head insulating member and the head bore of the implant head.

Due to the larger tightness of the press fit between the proximal pin of the head insulating member and the bore of the insertion bar, the insertion bar may be separated together with the head insulating member from the implant head. It can therefore be ensured that the head insulating member is removed together with the insertion bar after fixation of the suture at the bone tissue.

In some embodiments, the head bore of the implant head is a blind bore and comprises at least one longitudinally extending venting channel formed in a side wall of the blind bore, wherein the at least one venting channel has a larger axial length than the distal pin of the head insulating member.

By providing a venting channel, the blind bore of the implant head being closed by the distal pin of the head insulating member may outgas. For example, gas used for sterilization, such as ethylene oxide, may escape from the blind bore through the venting channel.

In some embodiments, the implant head comprises a proximal post, wherein the head bore of the implant head is at least partially formed in the proximal post of the implant head.

The proximal post may improve the radial stability at the region of the head insulating member.

In some embodiments, the implantation set comprises an insertion tube extending along the longitudinal axis from a proximal end to a distal end, and a tubular anchor sleeve having an outer thread and being made from magnesium or a magnesium alloy. The anchor sleeve is releasably connectable to-meaning that it can be attached to and detached from-a distal end of the insertion tube, wherein the insertion bar is slidably received through the insertion tube and the anchor sleeve such that the insertion tube and the anchor sleeve are axially (lengthwise) movable with respect to the insertion bar.

After placing the implant head at the bone, the anchor sleeve can be advanced towards the implant head by moving the insertion tube relative to the insertion bar towards the implant head. Once the anchor sleeve has reached the bone, the anchor sleeve may be screwed into the bone, thereby securing the implant head indirectly through the sutures that are threaded through the implant head and become blocked between the anchor sleeve and the bone during the screwing-in movement. After securing the implant head and the anchor sleeve in the bone, the insertion tube and the insertion bar may be separated from the implant head and the anchor sleeve, leaving the implant head and the anchor sleeve mounted in the bone.

In some embodiments, the insulating member further comprising at least one anchor insulating member arranged between the insertion tube and the anchor sleeve, the at least one anchor insulating member being configured to at least partially interrupt electric contact between the anchor sleeve and the insertion tube.

By providing an anchor insulating member between the insertion tube and the anchor sleeve, direct electric contact between the insertion tube and the anchor insulating member can be reduced or interrupted. An effective formation of a galvanic cell between the insertion tube and the anchor sleeve can be prevented and galvanic corrosion of the magnesium-based anchor sleeve may therefore significantly be reduced or avoided.

In some embodiments, the anchor insulating member or at least one of the anchor insulating members comprises an electrically insulating coating applied to at least one of: a portion of the insertion tube interfacing the anchor sleeve, and a portion of the anchor sleeve interfacing the insertion tube.

An electrically insulating coating may be used as an effective way to interrupt electric contact between the anchor sleeve and the insertion tube, as the relevant parts of the insertion tube and anchor sleeve that may come into direct contact with one another during arthroscopic insertion may selectively be covered by the coating. The electrically insulating coating provides enhanced flexibility as the coating may selectively be applied to a portion of the anchor sleeve or to a portion of the insertion tube or, if desired, to both a portion of the anchor sleeve and a portion of the insertion tube.

In a variant, the electrically insulating coating is applied to at least 80% of the surface of the insertion tube interfacing the anchor sleeve, or at least 80% of the surface of the anchor sleeve interfacing the insertion tube.

Preferably, the electrically insulating coating is applied to all the surface of the insertion tube interfacing the anchor sleeve, or all the surface of the anchor sleeve interfacing the insertion tube.

Covering all the interfacial surface with an insulating coating ensures an optimal insulation effect.

In some embodiments, the electrically insulating coating is selected from the group consisting of aluminum oxide, hydroxyl apatite, a lubricant varnish, parylene, diamond-like carbon, a electrolytic oxidized plasma coating of the insertion tube or portions thereof, and combinations thereof, whereby aluminum oxide is preferred.

These coatings effectively prevent galvanic corrosion, thereby reducing the risk of bubble formation during insertion. Furthermore, depending on the material chosen, they can serve a dual purpose, for example, by additionally promoting a biological response or providing a low-friction surface.

It has been demonstrated that the insertion tube made of surgical steel and the anchor sleeve made of magnesium or magnesium alloys or parts of the insertion tube or anchor sleeve can be effectively coated with the aforementioned coatings without negatively impacting the mechanical properties of the insertion tube or the anchor sleeve. On the contrary, the excellent properties can even be further enhanced. Furthermore, these biocompatible coatings have an excellent oxidation resistance and chemical resistance to various media including blood.

Preferably, the electrically insulating coating comprises aluminum oxide, for which excellent results were achieved. Aluminum oxide forms a stable, protective oxide layer and can effectively prevent galvanic corrosion, thereby minimizing the risk of gas accumulation and bubble formation during arthroscopic insertion. Furthermore, it reduces the risk of adverse reactions and promotes a favorable biological response.

Alternatively, the electrically insulating coating comprises hydroxyl apatite, which not only effectively prevents galvanic corrosion but also promotes bone bonding and integration with biological tissues.

Alternatively, the electrically insulating coating comprises lubricant varnish that can be applied easily and provides effective electrical insulation combined with and a low-friction surface.

Alternatively, the electrically insulating coating comprises parylene that has a high dielectric strength providing good electrical insulation in thin layers. Since it can be applied in very thin layers, it can be particularly utilized in geometrically complex areas of the anchor sleeve.

Alternatively, the electrically insulating coating comprises diamond-like carbon, which creates a robust protective layer that effectively mitigates galvanic corrosion.

Alternatively, the electrically insulating coating is formed by plasma electrolytic oxidation forming a ceramic-like coating that prevents galvanic corrosion.

The electrically insulating coating may have a thickness between 3 μm and 150 μm, preferably between 20 μm and 100 μm, more preferably between 30 μm and 50 μm.

A thickness of the coating in this range ensures a sufficient electrical insulation, while at the same time maintaining a smooth fit between the anchor sleeve and the insertion tube.

The electrically insulating coating may comprise a plurality of pores, and the anchor insulating member may further comprise a polymeric sealing on top of the electrically insulating coating. By closing a plurality of the pores of the electrically insulating coating with a polymeric sealing, the electrical insulation ability of the anchor insulating member is improved compared to a coating without additional polymeric sealing. Such a polymeric sealing can be applied easily to a variety of electrically insulating coatings.

The polymeric sealing may comprise a biocompatible polymer, such as polydimethylsiloxane, polyethylene glycol, poly(L-lactic acid), polyurethane, polyether ether ketone.

Preferably, the polymeric sealing comprises parylene, for which excellent results were achieved.

Parylene can be applied easily by chemical vapor deposition (CVD) or tip coating, which allows coating of complex geometries of the insertion tube or anchor sleeve. Due to its high dielectric strength, a parylene sealing ensures good electrical insulation in thin layers.

In a preferred embodiment, a parylene sealing is used in combination with an aluminum oxide electrically insulating coating. This combination yields excellent results for preventing galvanic corrosion.

The electrically insulating coating may be applied to a radial surface portion of the insertion tube or to a radial surface portion of the anchor sleeve.

In some embodiments, the implant head is a compression screw. The term compression screw relates to an orthopedic implant designed to stabilize and compress bone fragments to promote healing. Compression screws are characterized by their mechanical strength, ability to generate interfragmentary compression, and compatibility with surrounding bone.

In some embodiments, the implant head is an interference screw. The term interference screw relates to an orthopedic implant used primarily to secure soft tissue grafts within bone tunnels, such as in anterior cruciate ligament reconstruction. They have a threaded, screw-like design that allows them to be driven directly into bone, securing the graft in place. Interference screws are characterized by their ability to provide strong fixation through direct compression of the graft against the bone, ensuring stability during healing.

### BRIEF DESCRIPTION OF THE DRAWINGS

The present invention will be explained in more detail, by way of exemplary embodiments, with reference to the schematic drawings, in which:

FIG. 1 shows an axial cut view of a portion of an embodiment of an implantation set including an implant device in form of a suture anchor and an insertion bar;

FIG. 2 shows a magnified cut view of a portion of the implantation set of FIG. 1 having an electrically insulating coating between a portion of the insertion bar and a portion of the suture anchor;

FIG. 3 shows an axial cut view of a second embodiment of an implantation set including an implant device in form of a suture anchor, an insertion bar, an insertion tube and an anchor sleeve;

FIG. 4 shows a magnified cut view of a portion of an embodiment of the implantation set of FIG. 3 having an electrically insulating coating between a portion of the insertion bar and a portion of the suture anchor;

FIG. 5 shows a magnified cut view of a portion of the implantation set of FIG. 3 having an electrically insulating coating between a portion of the insertion tube and a portion of the anchor sleeve;

FIG. 6 shows a magnified cut view of a portion of an embodiment of an implantation set having O-rings arranged between a portion of an insertion bar and a portion of an implant head;

FIG. 7 shows a magnified cut view of a portion of an embodiment of an implantation set in the region of an implant head;

FIG. 8 shows the implantation set of FIG. 7 rotated by 90° around the longitudinal axis in a partially cut view;

FIG. 9 shows a head insulating member of FIG. 7 and FIG. 8 in a perspective view;

FIG. 10 shows a perspective cut view of an embodiment of an implant head;

FIG. 11 shows a top view of the implant head of FIG. 10.

FIG. 12 shows an axial cut view of a portion of an embodiment of an implantation set including an insertion bar and an implant device in form of a compression screw;

FIG. 13 shows a magnified cut view of a portion of the implantation set of FIG. 12 having an electrically insulating coating between a portion of the insertion bar and a portion of the implant head;

### DETAILED DESCRIPTION OF EXEMPLARY EMBODIMENTS

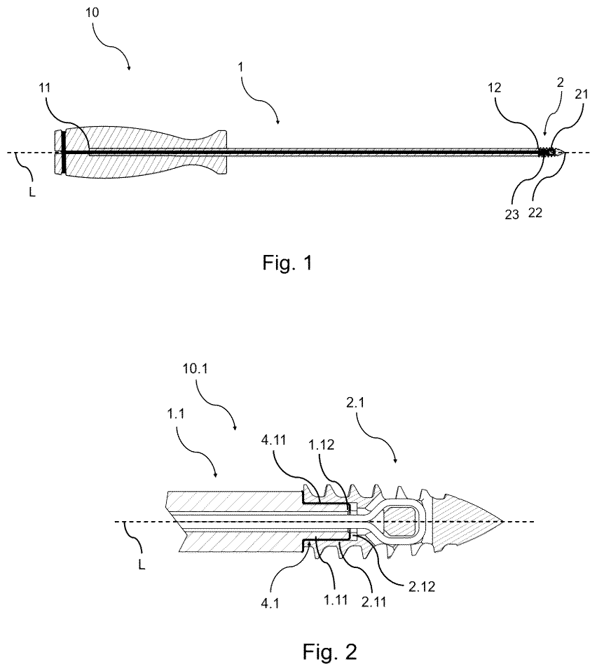

FIG. 1 shows an axial cut view of a portion of an embodiment of an implantation set 10 comprising an insertion bar 1 and an implant device with an implant head 2 in a connected state. The insertion bar 1 extends along a longitudinal axis L from a proximal end 11 to a distal end 12. The implant head 2 is made from magnesium or a magnesium alloy and is releasably connected to the distal end 12 of the insertion bar 1. The implant head 2 comprises eyelets 21 for receiving a suture therethrough. The implant head 2 is tapered towards a pointed tip 22 and comprises an outer thread 23 for being screwed into a bone tissue.

FIG. 2 shows a magnified cut view of a portion of an embodiment of an implantation set 10.1. The implantation set 10.1 corresponds to the one as shown in FIG. 1 in terms of the components of the insertion bar 1.1 and the implant head 2.1. The implantation set 10.1 comprises a head insulating member 4.1 formed by an electrically insulating coating 4.11 applied to a radial surface portion 1.11 and a bar axial stop surface 1.12 of the insertion bar 1.1 interfacing the implant head 2.1. In a variant, the electrically insulating coating 4.11 may additionally or alternatively be applied to a radial surface portion 2.11 and a head axial stop surface 2.12 of the implant head 2.1. The electrically insulating coating 4.11 comprises aluminum oxide and is applied by Atmospheric Plasma Spray (APS) after roughening the surface with sand blasting. In one variant, the target coating thickness is about 40 μm and in a second variant the target coating thickness is about 80 μm. In a further embodiment, a polymeric sealing is coated on top of the electrically insulating coating 4.11 by chemical vapor deposition (CVD) or tip coating to seal the pores of the electrically insulating coating. In a variant, this polymeric sealing comprises parylene.

Thus, the head insulating member 4.1 is configured to interrupt direct electrical contact between the implant head 2.1 and the insertion bar 1.1.

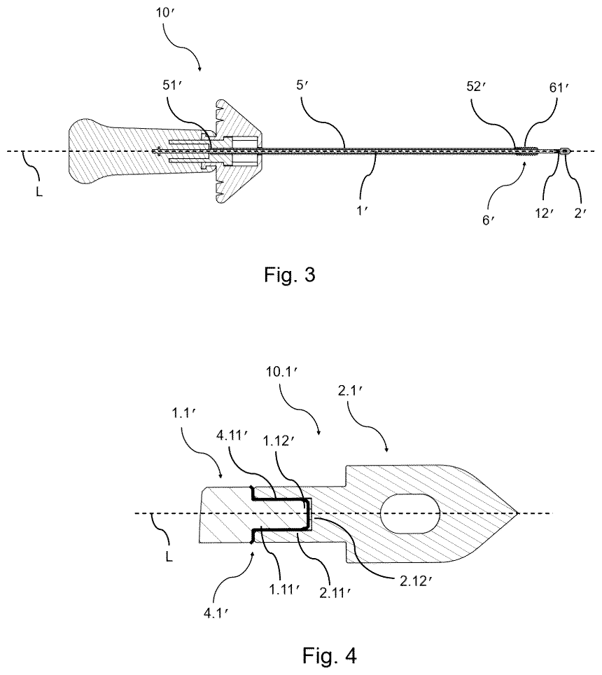

FIG. 3 shows an axial cut view of an embodiment of an implantation set 10′ comprising an insertion tube 5′ extending along a longitudinal axis L from a proximal end 51′ to a distal end 52′. The implantation set 10′ comprises an anchor sleeve 6′ being made from magnesium or a magnesium alloy which is releasably connected to the distal end 52′ of the insertion tube 5′. An insertion bar 1′ is slidably received through the insertion tube 5′ and the anchor sleeve 6′ such that the insertion tube 5′ and the anchor sleeve 6′ are axially movable with respect to the insertion bar 1′. The anchor sleeve 6′ comprises an outer thread 61′ for being screwed into a bone tissue. An implant head 2′ made from magnesium or a magnesium alloy is releasably connected to a distal end 12′ of the insertion bar 1′.

FIG. 4 shows a magnified cut view of a portion of an embodiment of an implantation set 10.1′ in the region of the implant head 2.1′. The implantation set 10.1′ corresponds to the one as shown in FIG. 3 in terms of the components of the insertion tube, the anchor sleeve, the insertion bar and the implant head. The implantation set 10.1′ comprises a head insulating member 4.1′ formed by an electrically insulating coating 4.11′ applied to a radial surface portion 1.11′ and a bar axial stop surface 1.12′ of the insertion bar 1.1′ interfacing the implant head 2.1′. In a variant, the electrically insulating coating 4.11′ may additionally or alternatively be applied to a radial surface portion 2.11′ and a head axial stop surface 2.12′ of the implant head 2.1′. The electrically insulating coating 4.11′ comprises aluminum oxide and is applied by Atmospheric Plasma Spray (APS) after roughening the surface with sand blasting. In one variant, the target coating thickness is between 20-60 μm, in particular 40 μm, and in a second variant the target coating thickness is about 80 μm. In a further variant, a polymeric sealing is coated on top of the electrically insulating coating 4.11′ by chemical vapor deposition (CVD) or tip coating to seal the pores of the electrically insulating coating. In a variant, this polymeric sealing comprises parylene.

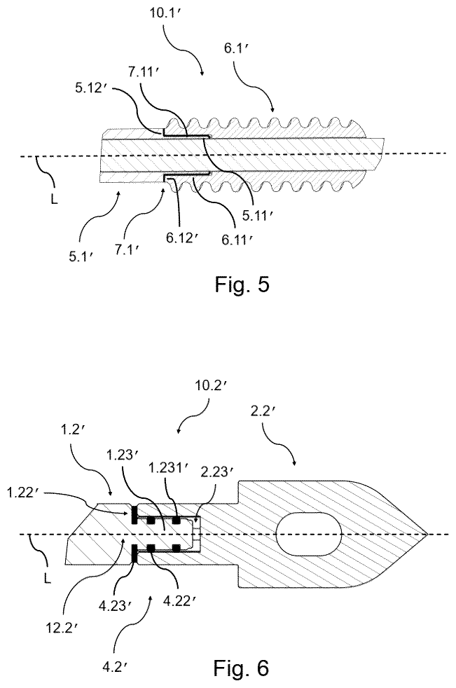

FIG. 5 shows a magnified cut view of a portion of the implantation set 10.1′ of FIG. 3 in the region of the anchor sleeve 6.1′. The implantation set 10.1′ comprises an anchor insulating member 7.1′ formed by an electrically insulating coating 7.11′ applied to a radial surface portion 5.11′ and an axial stop surface 5.12′ of the insertion tube 5.1′ interfacing the anchor sleeve 6.1′. In a variant, the electrically insulating coating 7.11′ may additionally or alternatively be applied to a radial surface portion 6.11′ and an axial stop surface 6.12′ of the anchor sleeve 6.1′. The electrically insulating coating 7.11′ comprises aluminum oxide and is applied by Atmospheric Plasma Spray (APS) after roughening the surface with sand blasting. In one variant, the target coating thickness is between 20-60 μm, in particular 40 μm, and in a second variant the target coating thickness is about 80 μm. In a further variant, a polymeric sealing is coated on top of the electrically insulating coating 7.11′ by chemical vapor deposition (CVD) or tip coating to seal the pores of the electrically insulating coating. In a variant, this polymeric sealing comprises parylene.

FIG. 6 shows a magnified cut view of a portion of an embodiment of an implantation set 10.2′ corresponding to the one as shown in FIG. 3 in terms of the components of the insertion tube, the anchor sleeve, the insertion bar and the implant head. The insertion bar 1.2′ comprises at its distal end 12.2′ a bar pin 1.23′ protruding a bar axial stop surface 1.22′. The bar pin 1.23′ is received in a head bore 2.23′ of the implant head 2.2′. The bar pin 1.23′ comprises two circumferential recesses 1.231′ each partially receiving a rubber O-ring 4.22′ such that the O-rings 4.22′ are arranged between the bar pin 1.23′ and a side wall of the head bore 2.23′. Furthermore, an insulating disc 4.23′ with a central aperture is arranged at the bar axial stop surface 1.22′. The insulating disc 4.23′ is made of nitrile butadiene rubber (NBR). The O-rings 4.22′ and the insulating disc 4.23′ with a central aperture therefore serve as head insulating members 4.2′ preventing direct electric contact between the insertion bar 1.2′ and the implant head 2.2′.

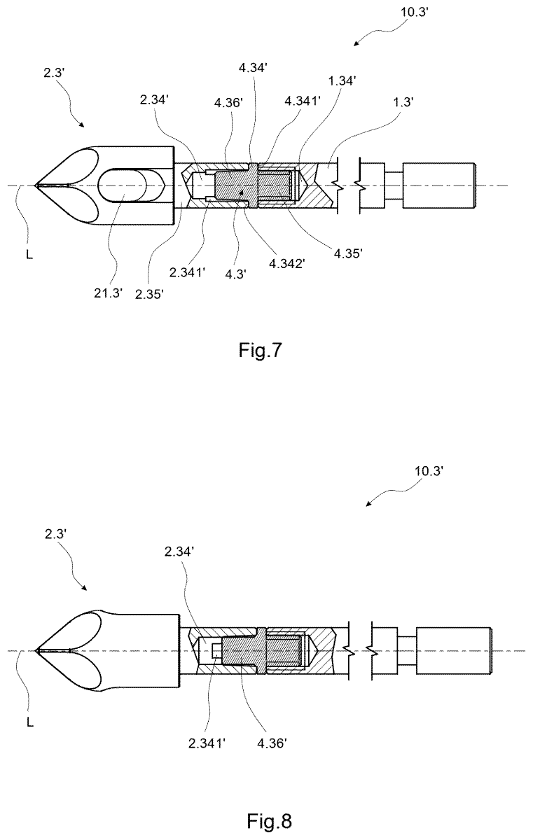

FIG. 7 shows a partially cut view of an embodiment of an implantation set 10.3′ in the region of the implant head 2.3′. The implantation set 10.3′ corresponds to the one as shown in FIG. 3 in terms of the components of the insertion tube, the anchor sleeve, the insertion bar and the implant head. The implant head 2.3′ comprises an eyelet 21.3′ for receiving a suture therethrough. The implantation set 10.3′ comprises a head insulating member 4.3′ having a stop disc 4.34′ with a proximal first axial stop surface 4.341′ for the insertion bar 1.3′ and a distal second axial stop surface 4.342′ for the implant head 2.3′. A proximal pin 4.35′ protrudes from the stop disc 4.34′ in a proximal direction towards the insertion bar 1.3′. The insertion bar 1.3′ comprises a bore 1.34′ in which the proximal pin 4.35′ is received by a screw connection. A distal pin 4.36′ protrudes from the stop disc 4.34′ in a distal direction towards the implant head 2.3′. The implant head 2.3′ comprises a bore 2.34′ in which the distal pin 4.36′ is received by a press fit. The bore 2.34′ of the implant head 2.3′ is a blind bore and comprises two venting channels 2.341′ in the side wall of the bore 2.34′. The axial length of the venting channels 2.341′ is larger than the axial length of the distal pin 4.36′, allowing outgassing of the bore 2.34′ through the venting channels 2.341′. The implant head 2.3′ comprises a proximal post 2.35′ in which the bore 2.34′ is formed. The head insulating member 4.3′ is made of polyether ether ketone and interrupts direct electric contact between the implant head 2.3′ and the insertion bar 1.3′.

FIG. 8 shows the implantation set 10.3′ of FIG. 7 rotated by 90° around the longitudinal axis L in a partially cut view. In this view, only one of the venting channels 2.341′ is visible. Furthermore, the press fit between the distal pin 4.36′ and the bore 2.34′ of the implant head 2.3′ can be recognized.

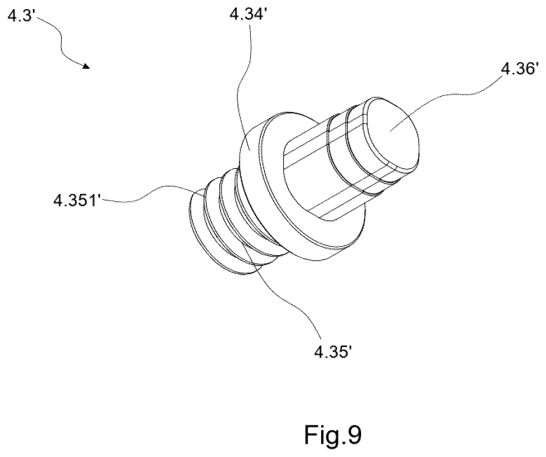

FIG. 9 shows the head insulating member 4.3′ of FIGS. 7 and 8 in a perspective view. The distal pin 4.36′ distally protruding from the stop disc 4.34′ is stepwise tapered and dimensioned to provide a press fit with the corresponding bore of the head insulating member. The proximal pin 4.35′ proximally protruding from the stop disc 4.34′ comprises an outer thread 4.351′ such that the proximal pin 4.35′ can engage in a screw connection with the corresponding bore of the insertion bar having an inner thread. The head insulating member 4.3′ therefore has a shape of a double pin with a central stop disc 4.34′.

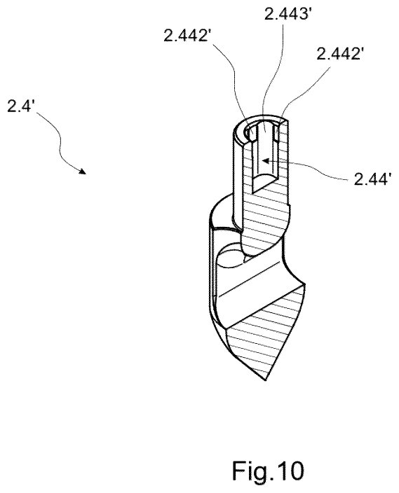

FIG. 10 shows a perspective cut view of an embodiment of an implant head 2.4′. The implant head 2.4′ comprises a bore 2.44′ with four planar radial stop surfaces 2.442′ (only two radial stop surfaces are visible in FIG. 10) and partially circular sections 2.443′ between the planar radial stop surfaces 2.442′.

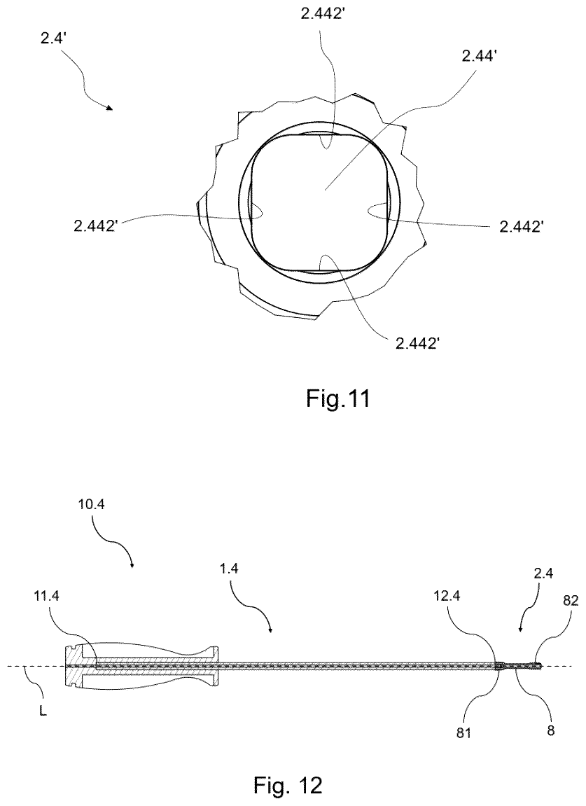

FIG. 11 shows a top view of the implant head 2.4′ of FIG. 10 from the side of the bore 2.44′. The four planar radial stop surfaces 2.442′ are arranged in a rectangular fashion such that neighboring planar radial stop surfaces 2.442′ are perpendicularly oriented to each other. The bore 2.44′ with the four planar radial stop surfaces 2.442′ is dimensioned to engage with a distal end of an insertion bar or with a pin of a head insulating member with a circular cross-section by a press fit such that the distal end of the insertion bar or the pin of a head insulating member abut the four planar radial stop surfaces 2.442′.

FIG. 12 shows an axial cut view of a portion of an embodiment of an implantation set 10.4 comprising an insertion bar 1.4 extending along a longitudinal axis L from a proximal end 11.4 to a distal end 12.4. The implantation set 10.4 comprises a compression screw 8 as implant head 2.4 made from magnesium or a magnesium alloy which is releasably connected to the distal end 12.4 of the insertion bar 1.4. The compression screw 8 comprises two outer threads for being screwed into a bone tissue, one proximal thread 81 at the proximal end of the compression screw 8 relative to the insertion bar 1.4 and one distal thread 82 at the distal end of the compression screw 8 relative to the insertion bar 1.4.

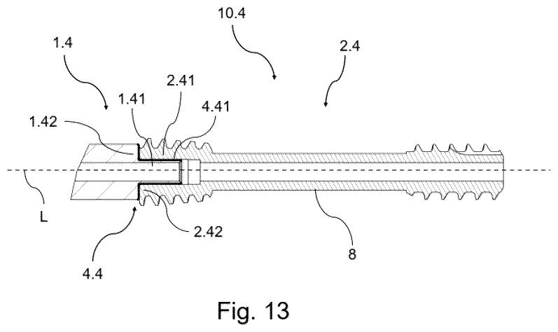

FIG. 13 shows a magnified cut view of a portion of the implantation set 10.4 in the region of the implant head 2.4. The implantation set 10.4 corresponds to the one as shown in FIG. 12 in terms of the components of the insertion bar 1.4 and the implant head 2.4 in the form of a compression screw 8. The implantation set 10.4 comprises a head insulating member 4.4 formed by an electrically insulating coating 4.41 applied to a radial surface portion 1.41 and a bar axial stop surface 1.42 of the insertion bar 1.4 interfacing the implant head 2.4. In a variant, the electrically insulating coating 4.41 may additionally or alternatively be applied to a radial surface portion and a head axial stop surface of the implant head 2.4. The electrically insulating coating 4.41 comprises aluminum oxide and is applied by Atmospheric Plasma Spray (APS) after roughening the surface with sand blasting. In one variant, the target coating thickness is between 20-60 μm, in particular 40 μm, and in a second variant the target coating thickness is about 80 μm. In a further embodiment, a polymeric sealing is coated on top of the electrically insulating coating 4.41 by chemical vapor deposition (CVD) or tip coating to seal the pores of the electrically insulating coating. In a variant, this polymeric sealing comprises parylene.

Thus, the head insulating member 4.4 is configured to interrupt direct electrical contact between the implant head 2.4 and the insertion bar 1.4.

### LIST OF REFERENCE SYMBOLS

10, 10′, 10.1, 10.4
10.1′, 10.2′, 10.3′ Implantation set
1, 1′, 1.1,
1.1′, 1.2′, 1.3′, 1.4 Insertion bar
1.11, 1.11′, 1.41 Radial surface portion
1.12, 1.12′, 1.22′, 1.42 Bar axial stop surface
11, 11.4 Proximal end of the insertion bar
12, 12.2′, 12.4 Distal end of the insertion bar
1.23′ Bar pin
1.231′ Recess
1.34′ Bore
2, 2′, 2.1, 2.1′,
2.2′, 2.3′, 2.4, 2.4′ Implant head
21, 21.3′ Eyelet
22 Pointed tip
23 Outer thread
2.11, 2.11′, 2.41 Radial surface portion
2.12, 2.12′, 2.42 Head axial stop surface
2.23′ Head bore
2.34′, 2.44′ Bore
2.341′ Venting channel
2.35′ Proximal post
2.442′ Planar radial stop surface
2.443′ Partially circular section
4.1, 4.1′, 4.2′, 4.3′, 4.4 Head insulating member
4.11, 4.11′, 4.41 Electrically insulating coating
4.22′ O-ring
4.23′ Insulating disc
4.34′ Stop disc
4.341′ Proximal first axial stop surface
4.342′ Distal second axial stop surface
4.35′ Proximal pin
4.351′ Outer thread
4.36′ Distal pin
5′, 5.1′ Insertion tube
51′ Proximal end of the insertion tube
52′ Distal end of the insertion tube
5.11′ Radial surface portion
5.12′ Axial stop surface
6′, 6.1′ Anchor sleeve
61′ Outer thread
6.11′ Radial surface portion
6.12′ Axial stop surface
7.1′ Anchor insulating member
7.11′ Electrically insulating coating
8 Compression screw
81 Proximal thread
82 Distal thread
L Longitudinal axis

1. An implantation set comprising an insertion device, an implant device, and one or more insulating member(s),
the insertion device including an insertion bar extending along a longitudinal axis from a proximal end to a distal end and being configured for inserting the implant device into bone;
the implant device including an implant head made from magnesium or a magnesium alloy and being releasably connectable to the distal end of the insertion bar, thereby bringing the insertion device and the implant device temporarily into a connected state,
the insulating member including one or more head insulating member(s) arranged between the insertion bar and the implant head in the connected state, thereby at least partially interrupting direct electric contact between the implant head and the insertion bar.

2. The implantation set according to claim 1, wherein one head insulating member comprises an electrically insulating coating applied to a portion of the insertion bar interfacing the implant head and/or a portion of the implant head interfacing the insertion bar.

3. The implantation set according to claim 2, wherein the electrically insulating coating is selected from the group consisting of aluminum oxide, zirconia, yttria-stabilized zirconia, chromium oxide, titanium dioxide, magnesium aluminate spinel, hafnium oxide, lanthanum-based oxides, calcium zirconate, silicon oxide, hydroxyl apatite, a lubricant varnish, parylene, diamond-like carbon, a electrolytic oxidized plasma coating, and combinations thereof.

4. The implantation set according to claim 2, wherein the electrically insulating coating has a thickness between 3 μm and 150 μm.

5. The implantation set according to claim 2, wherein the electrically insulating coating comprises a plurality of pores, and the head insulating member further comprises a polymeric sealing on top of the electrically insulating coating.

6. The implantation set according to claim 2, wherein the electrically insulating coating is applied to a radial surface portion of the insertion bar and/or to a radial surface portion of the implant head.

7. The implantation set according to claim 1, wherein the implant device is selected from the group consisting of a suture anchor, a fixation pin, a punching tip, and a screw.

8. The implantation set according to claim 7, wherein the implant device is a suture anchor for attaching soft tissue to bone.

9. The implantation set according to claim 1, wherein the insertion bar comprises a bar axial stop surface and the implant head comprises a head axial stop surface, wherein one head insulating member is arranged between the bar axial stop surface and the head axial stop surface.

10. The implantation set according to claim 9, wherein the head insulating member arranged between the bar axial stop surface and the head axial stop surface comprises an electrically insulating coating applied to at least one of: the bar axial stop surface, the head axial stop surface.

11. The implantation set according to claim 1, wherein the insertion bar comprises a bar pin at its distal end, protruding from the bar axial stop surface, wherein the implant head comprises a head bore dimensioned to receive the bar pin, wherein, in the connected state, at least one head insulating member is arranged between the bar pin and a side wall of the head bore.

12. The implantation set according to claim 11, wherein the bar pin is dimensioned to engage with the head bore by a press fit.

13. The implantation set according to claim 12, wherein the implant head is a screw head including a head bore having one or more planar radial stop surfaces, and the insertion bar or the bar pin comprises a distal end portion including engagement elements dimensioned to engage with the radial stop surfaces for transferring a torque to the screw head.

14. The implantation set according to claim 1, wherein one head insulating member comprises a stop disc with a proximal circumferential surface forming a first axial stop surface for the insertion bar and a distal circumferential surface forming a second axial stop surface for the implant head.

15. The implantation set according to claim 1, further comprising an insertion tube extending along the longitudinal axis from a proximal end to a distal end, a tubular anchor sleeve having an outer thread and being made from magnesium or a magnesium alloy, wherein the anchor sleeve is releasably connectable to a distal end of the insertion tube, wherein the insertion bar is slidably received through the insertion tube and the anchor sleeve such that the insertion tube and the anchor sleeve are axially movable with respect to the insertion bar.

16. The implantation set according to claim 15, the insulating member further comprising at least one anchor insulating member arranged between the insertion tube and the anchor sleeve, the at least one anchor insulating member being configured to at least partially interrupt electric contact between the anchor sleeve and the insertion tube.

17. The implantation set according to claim 16, wherein at least one anchor insulating member comprises an electrically insulating coating applied to at least one of: a portion of the insertion tube interfacing the anchor sleeve, a portion of the anchor sleeve interfacing the insertion tube.

18. The implantation set according to claim 17, wherein the electrically insulating coating is selected from the group consisting of aluminum oxide, zirconia, yttria-stabilized zirconia, chromium oxide, titanium dioxide, magnesium aluminate spinel, hafnium oxide, lanthanum-based oxides, calcium zirconate, silicon oxide, hydroxyl apatite, a lubricant varnish, parylene, diamond-like carbon, a electrolytic oxidized plasma coating of the insertion tube or portions thereof, and combinations thereof.

19. The implantation set according to claim 1, wherein the implant head is a compression screw.

20. The implantation set according to claim 1, wherein the implant head is an interference screw.
